(19) United States
(12) Patent Application Publication
(10) Pub. No.: US 2010/0247512 A1
(43) Pub. Date: Sep. 30, 2010

(54) Pericellular Collagenase Directs the 3-Dimensional Development of White Adipose Tissue

(75) Inventors: Stephen Weiss, Ann Arbor, MI (US); Tae-Hwa Chun, Ann Arbor, MI (US)

Correspondence Address: MARSHALL, GERSTEIN & BORUN LLP, 233 SOUTH WACKER DRIVE, 6300 WILLIS TOWER, CHICAGO, IL, 60606-6357, US

(73) Assignee: The Regents of the University of Michigan, Ann Arbor, MI (US)

(21) Appl. No.: 12/293,984

(22) PCT Filed: Apr. 2, 2007
(86) PCT No.: PCT/US07/08288

§ 371 Date: Jun. 18, 2010

Related U.S. Application Data
(60) Provisional application No. 60/787,768, filed on Mar. 31, 2006.

(51) Int. Cl. A61K 38/46; A61P 3/00

(52) U.S. Cl. 424/94.67

(57) ABSTRACT

Methods are provided for regulating fat storage, carbohydrate metabolism and fatty acid synthesis using modulators of membrane type matrix metalloproteinase 1 (MT1-MMP).

### RELATED APPLICATIONS

This application claims priority of U.S. Provisional Patent Application No. 60/787,768 filed Mar. 31, 2006, the disclosure of which is incorporated herein in its entirety.

### GRANT FUNDING

This work was supported by National Institutes of Health grant No. R01 CA088308.

### BACKGROUND

White adipose tissue (WAT) plays a key role in regulating energy homeostasis by not only functioning as the primary depot for energy stores, but also by secreting key signaling molecules that impact on multiple target organ systems (Flier, 2004). During development, mesenchymal stem cells that populate the primordial fat pad are induced to commit to the adipocyte lineage (Rosen and Spiegelman, 2000). Within a 3-D organoid-like environment that is rich in extracellular matrix (ECM) macromolecules, the adipocyte precursors (i.e., preadipocytes) undergo a sequential differentiation program to form lipid-laden adipocytes that respond to insulin signaling, synthesize and store triglyceride deposits, and express functionally important adipokines (Napolitano, 1963; Rosen and Spiegelman, 2000; Saltiel and Kahn, 2001). To orchestrate this cellular transformation, preadipocytes engage a network of multiple transcription factors that regulate both the adipogenic program as well as the final mix of gene products expressed in the mature adipocyte (Rosen and Spiegelman, 2000). However, the epigenetic regulators that control the expression of this transcription program in the in vivo setting remain largely uncharacterized.

MT1-MMP is the prototypical member of a subgroup of membrane-tethered proteinases that belong to the matrix metalloproteinase (MMP) gene family (Itoh and Seiki, 2006). Synthesized as a proenzyme, the MT1-MMP zymogen undergoes activation following intracellular processing and is displayed on the cell surface as an active proteinase that can degrade a wide variety of cell-associated, soluble or ECM-derived targets (e.g., Hiraoka et al., 1998; Hotary et al., 2003; Chun et al., 2004; Lehti et al., 2005; Itoh and Seiki, 2006). The functional consequences of MT1-MMP-mediated hydrolytic events in vivo are largely undefined, but it is noteworthy that MT1-MMP-null mice display a poorly characterized failure to thrive and a markedly shortened lifespan (Holmbeck et al., 1999; Zhou et al., 2000).

### SUMMARY OF THE INVENTION

Methods are provided for regulating fat storage comprising the step of administering a modulator of membrane-tethered metalloproteinase 1 (MT1-MMP) in an amount effective to modulate MT1-MMP activity. In one aspect, the modulator is an inhibitor of MT1-MMP and fat storage in inhibited. In another aspect, the modulator is an activator of MT1-MMP and fat storage is increased.

In one embodiment, methods are provided for increasing fat storage comprising the step of administering an amount of membrane-tethered metalloproteinase 1 (MT1-MMP) in an amount effective to increase fat storage. In another embodiment, methods of regulating carbohydrate metabolism are provided comprising the step of administering a modulator of membrane-tethered metalloproteinase 1 (MT1-MMP) in an amount effective to modulate carbohydrate metabolism. In one aspect, the modulator is an inhibitor of MT1-MMP and carbohydrate metabolism in inhibited. In another aspect, the modulator is an activator of MT1-MMP and carbohydrate metabolism is increased.

In another embodiment, methods are provide for increasing carbohydrate metabolism comprising the step of administering an amount of membrane-tethered metalloproteinase 1 (MT1-MMP) in an amount effective to increase carbohydrate metabolism. In yet another embodiment, methods are provided for regulating fatty acid synthesis comprising the step of administering a modulator of membrane-tethered metalloproteinase 1 (MT1-MMP) in an amount effective to modulate fatty acid synthesis. In one aspect, the modulator is an inhibitor of MT1-MMP and fatty acid synthesis in inhibited. In another aspect, the modulator is an activator of MT1-MMP and fatty acid synthesis is increased.

In still another embodiment, methods are provided for increasing fatty acid synthesis comprising the step of administering an amount of membrane-tethered metalloproteinase 1 (MT1-MMP) in an amount effective to increase fatty acid synthesis.

In another embodiment, methods are provided for regulating fat storage comprising the step of administering a modulator of a membrane-tethered metalloproteinase 1 (MT1-MMP) downstream pathway component in an amount effective to modulate activity of said component. In one aspect, the component is one or more transcription factors. In other aspects, the one or more transcription factors is selected from the group consisting of C/EPBβ, C/EBPα, PPARα AND NRFB1. In still other aspects, the modulator increases activity of said component or in the alternative, the modulator decreases activity of said component.

### DETAILED DESCRIPTION OF THE INVENTION

Herein, MT1-MMP is shown to play a required role in regulating WAT development and function. In the absence of MT1-MMP, the transcription factor cascades that underlie adipocyte maturation are disrupted with consequent effects on WAT function. Unexpectedly, it was found that normal adipocyte maturation only proceeds when coordinated with the tandem MT1-MMP-dependent remodeling of the 3-D type I collagen scaffolding which dominates primordial white fat deposits. In the absence of MT1-MMP, preadipocytes are unable to mount a required collagenolytic response during WAT development, and are entrapped within a dense meshwork of collagen fibrils that disrupt both cell cytoarchitecture and cell signaling programs critical to adipogenesis. These findings serve to identify adipocyte MT1-MMP as a heretofore unsuspected, 3-D matrix-specific regulator of WAT development and function.

Accordingly, methods are provided for regulating fat storage comprising the step of administering a modulator of membrane-tethered metalloproteinase 1 (MT1-MMP) in an amount effective to modulate MT1-MMP activity.

The term “membrane-tethered metalloproteinase 1 (MT1-MMP)” as used herein refers full length mature proteins, as well as enzymatically active fragments and variants thereof, proproteins, and preproproteins, of any species. It is noted that MT1-MMP is also known in the art as matrix metalloprotein 14 (MMP-14) and membrane-type matrix metalloprotein 1 (MT-MMP1).

The term “variants” refers to naturally-occurring proteins, as well as proteins modified by man which maintain the same, or essentially the same, enzymatic activity as a naturally-occurring protein.

The terms “effective amount” and “therapeutically effective amount” each refer to the amount of an of an MT1-MMP used to support an observable level of one or more biological activities of an MT1-MMP.

The term “modulator” as used herein refers to a compound that either increases or decreases the enzymatic activity of MT1-MMP and includes, without limitation, proteins, peptides, small molecules, antisense polynucleotides including small inhibitory RNA and other interfering RNAs, ribozymes, triplex-forming polynucleotides, and the like. Within the class of proteins and peptides, antibodies and binding fragments thereof are contemplated. Activators specifically include compounds that, for example, initiate a biochemical cascade that eventually leads to a more active form of MT1-MMP, or compounds that act to inhibit a naturally-occurring MT1-MMP inhibitor. Modulators that affect MT1-MMP activity act at a transcriptional level, a translational level, a post-translational level or at two or all three of these levels A modulator as contemplated by the invention acts directly on MT1-MMP or acts indirectly on MT1-MMP. A modulator that “acts indirectly” on MT1-MMP increases or decreases MT1-MMP activity by acting on a compound that in some way affects MT1-MMP activity and includes, without limitation, compounds that modulate an upstream activator or inhibitor of MT1-MMP activity. Such modulators of an upstream activator or inhibitor act at a transcriptional level, a translational level, a post-translational level or at two or all three of these levels. With knowledge of biochemical pathways in which MT1-MMP activity is a rate-limiting component, the invention further contemplates use of compounds that act, either directly or indirectly, on downstream compounds in a biochemical pathway that achieves the same result as if MT1-MMP activity were directly or indirectly modulated.

Accordingly, in one aspect, the modulator is an inhibitor of MT1-MMP and fat storage in inhibited. In another aspect, the modulator is an activator of MT1-MMP and fat storage is increased. Thus, in one embodiment, methods are provided for increasing fat storage comprising the step of administering an amount of membrane-tethered metalloproteinase 1 (MT1-MMP) in an amount effective to increase fat storage, and in another embodiment, methods are provided for decreasing fat storage comprising the step of administering an amount of membrane-tethered metalloproteinase 1 (MT1-MMP) in an amount effective to decrease fat storage.

In another embodiment, methods of regulating carbohydrate metabolism are provided comprising the step of administering a modulator of membrane-tethered metalloproteinase 1 (MT1-MMP) in an amount effective to modulate carbohydrate metabolism. In one aspect, the modulator is an inhibitor of MT1-MMP and carbohydrate metabolism in inhibited. In another aspect, the modulator is an activator of MT1-MMP and carbohydrate metabolism is increased.

In another embodiment, methods are provided for increasing fatty acid synthesis comprising the step of administering an amount of membrane-tethered metalloproteinase 1 (MT1-MMP) in an amount effective to increase fatty acid synthesis, as well as methods for decreasing fatty acid synthesis comprising the step of administering an amount of membrane-tethered metalloproteinase 1 (MT1-MMP) in an amount effective to decrease fatty acid synthesis.

### Exemplary MT1-MMP Proteins

Any MT1-MMP protein is contemplated as being useful in the methods of the invention, including, but not limited to, those proteins set out in Genbank Accession No: AAH64803 (human), Genbank Accession No: P50281 (human), Genbank Accession No: NP_{—}004986 (human), Genbank Accession No: Q9XT90 (pig), Genbank Accession No: NP_{—}999404 (pig), Genbank Accession No: Q95220 (rabbit), Genbank Accession No: Q10739 (Norwegian rat), Genbank Accession No: NP_{—}112318 (Norwegian rat), Genbank Accession No: AAH72509 (Norwegian rat), Genbank Accession No: P53690 (house mouse), Genbank Accession No: NP_{—}032634 (house mouse), Genbank Accession No: AAH76638.1 (house mouse), Genbank Accession No: BAD99513 (Japanese medaka), Genbank Accession No: AAD30298 (cow), as well as other MT1-MMP proteins having the desired biological activity.

### Representative Inhibitors

Inhibitors of MT1-MMP are well known in the art and with increased understanding of modes of inhibition of these compounds, more inhibitors continue to be identified and/or synthesized. It will be understood by the skilled artisan that, with respect to naturally-occurring inhibitors, compounds that are capable of blocking the inhibitory effect of these compounds would be considered activators as defined herein. Inhibitors contemplated include, without limitation, any of the following. Naturally-occurring mammalian compounds are exemplified by the tissue inhibitors of metalloproteinases (TIMPs) which are a family of natural protein MMP inhibitors. (Toth et al., 2002). TIMP-1 is a poor inhibitor of MT1-MMP for to blocking collagenolysis and the ability, while TIMP-2 is an efficient MT1-MMP inhibitor that blocks collagenolysis (Rosenthal, et al., 2005). In addition, tissue inhibitor of metalloproteinase (TIMP)-2 which acts synergistically with synthetic matrix metalloproteinase (MMP) inhibitors but not with TIMP-4 to enhance the (Membrane type 1)-MMP-dependent activation of pro-MMP-2. (Toth et al., 2000). Naturally-occurring inhibitors also include testican 1 and 3, RECK, and the Cupin superfamily member MTCBP-1 (Zhai, et al., 2005). Naturally-occurring inhibitors from non-mammalian species are exemplified by Callysponginol sulfate A, from the marine sponge Callyspongia truncate. (Fugita, et al., 2003). Small molecule inhibitors are exemplified by hydroxamate MMP inhibitors, including known inhibitors such as BB-94, BB-2516, GM6001, and Ro31-9790 (Yamamoto, et al., (1998), as well as potent MMP inhibitors with a mercaptosulphide zinc-binding functionality that have been designed and synthesized. (Hurst, et al., 2005). Antibody and siRNA inhibitors are exemplified by those described by Muñoz-Nája, et al., (2006), and peptide inhibitors are exemplified by the synthetic furin inhibitor deconyl-Arg-Val-Lys-Arg-CMK (CMK), a synthetic peptidyl chloromethyl ketone, which inhibits the intracellular conversion of pro-MT1-MMP to its active form (Rosenthal, et al., 2005)

For clarity, the term “naturally occurring” or “native” when used in connection with biological materials refers to materials which are found in nature and are not manipulated by man except for possible purification. Similarly, “non-naturally occurring” or “non-native” “non-naturally occurring” or “non-native” as used herein refers to a material that is not found in nature or that has been structurally modified or synthesized by man.

### Formulations

The term “pharmaceutically acceptable carrier” or “physiologically acceptable carrier” as used herein refers to one or more formulation materials suitable for accomplishing or enhancing the delivery of an MT1-MMP.

In one aspect, MT1-MMP modulator pharmaceutical compositions comprise a therapeutically effective amount of a modulator with a pharmaceutically or physiologically acceptable formulation agent selected for suitability with the mode of administration. In another aspect, pharmaceutical compositions comprise a therapeutically effective amount of one or more modulators with a pharmaceutically or physiologically acceptable formulation agent selected for suitability with the mode of administration.

In various aspects, the pharmaceutical composition contain formulation materials for modifying, maintaining or preserving, for example, the pH, osmolarity, viscosity, clarity, color, isotonicity, odor, sterility, stability, rate of dissolution or release, adsorption or penetration of the composition. Suitable formulation materials include, but are not limited to, amino acids (such as glycine, glutamine, asparagine, arginine or lysine),lysine); antimicrobials, antimicrobials; antioxidants (such as ascorbic acid, sodium sulfite or sodium hydrogen-sulfite),hydrogen-sulfite); buffers (such as borate, bicarbonate, Tris-HCl, citrates, phosphates, phosphates or other organic acids),acids); bulking agents (such as mannitol or glycine),glycine); chelating agents (such as ethylenediamine tetraacetic acid (EDTA)), (EDTA)); complexing agents (such as caffeine, polyvinylpyrrolidone, beta-cyclodextrin or hydroxypropyl-beta-cyclodextrin), fillers, hydroxypropyl-beta-cyclodextrin); fillers; monosaccharides, disaccharides, monosaccharides; disaccharides; and other carbohydrates (such as glucose, mannose, or dextrins),mannose or dextrins); proteins (such as serum albumin, gelatin or immunoglobulins),immunoglobulins); coloring, flavoring and diluting agents, agents; emulsifying agents, agents; hydrophilic polymers (such as polyvinylpyrrolidone),polyvinylpyrrolidone); low molecular weight polypeptides, polypeptides; salt-forming counterions (such as sodium),sodium); preservatives (such as benzalkonium chloride, benzoic acid, salicylic acid, thimerosal, phenethyl alcohol, methylparaben, propylparaben, chlorhexidine, sorbic acid or hydrogen peroxide),peroxide); solvents (such as glycerin, propylene glycol or polyethylene glycol),glycol); sugar alcohols (such as mannitol or sorbitol),sorbitol); suspending agents, agents; surfactants or wetting agents (such as pluronics, PEG, sorbitan esters, polysorbates such as polysorbate 20, polysorbate 80, triton, tromethamine, lecithin, cholesterol, tyloxapal),tyloxapal); stability enhancing agents (sucrose or sorbitol), (such as sucrose or sorbitol); tonicity enhancing agents (such as alkali metal halides (preferablyhalides, preferably sodium or potassium chloride), mannitol sorbitol), delivery vehicles, diluents, chloride, mannitol sorbitol); delivery vehicles; diluents; excipients and/or pharmaceutical adjuvants. (Remington's Pharmaceutical Sciences, 18th Edition, A. R. Gennaro, ed., Mack Publishing Company [1990]). (1990).

Pharmaceutical composition are optimized by one skilled in the art depending upon, for example, the intended route of administration, delivery format, and desired dosage. See, for example, Remington's Pharmaceutical Sciences, supra.

In alternative aspects, a vehicle or carrier in a pharmaceutical composition is either aqueous or non-aqueous in nature. For example, a suitable vehicle or carrier is water, neutral buffered saline or saline mixed with serum albumin for injection. In other aspects, exemplary pharmaceutical compositions comprise Tris buffer of about pH 7.0-8.5, or acetate buffer of about pH 4.0-5.5, which may further include sorbitol or a suitable substitute therefor. In one aspect, modulator compositions may be prepared for storage by mixing the selected composition having the desired degree of purity with optional formulation agents (Remington's Pharmaceutical Sciences, supra) in the form of a lyophilized cake or an aqueous solution.

The formulation components are present in concentrations that are acceptable for the route of administration. Typically, buffers are used to maintain the composition at physiological pH or at a pH in the range of from about 5 to about 8.

For parenteral administration, the therapeutic compositions may be in the form of a pyrogen-free, parenterally acceptable aqueous solution comprising the desired modulator in a pharmaceutically acceptable vehicle. An exemplary vehicle for parenteral injection is sterile distilled water. Yet another preparation can involve the formulation of the modulator with an agent, such as injectable microspheres, polymeric compounds (such as polylactic (polylactic acid, acid or polyglycolic acid), beads, or liposomes, that provides for the controlled or sustained release of the modulator.

In pharmaceutical compositions formulated for inhalation, the modulator or modulators may be formulated as a dry powder, optionally with a propellant for aerosol delivery. In another embodiment, modulator solutions may be nebulized. Pulmonary administration is described in PCT application No. PCT/US94/001875.

Pharmaceutical formulations may be prepared for oral administration. In one embodiment modulators are formulated with or without those carriers customarily used in the compounding of solid dosage forms such as tablets and capsules. For example, a capsule may be designed to release the modulator at the point in the gastrointestinal tract when bioavailability is maximized and pre-systemic degradation is minimized. Additional agents can be included to facilitate absorption of the modulator including, without limitation diluents, flavorings, low melting point waxes, vegetable oils, lubricants, suspending agents, tablet disintegrating agents, and binders.

In others aspects, modulator compositions are prepared in sustained- or controlled-delivery formulations. Techniques for formulating sustained- or controlled-delivery means, such as liposome carriers, microparticles or porous beads and depot injections, are also known to those skilled in the art as described in PCT Application No. PCT/US93/00829. Additional examples of sustained-sustained-release preparations include polyesters, hydrogels, polylactides (U.S. Pat. No. 3,773,919 and EP 3,773,919, EP 58,481),058,481), copolymers of L-L-glutamic acid and gamma ethyl-L-glutamate (Sidman et al., Biopolymers, 22:547-556 (1983)), poly (2-hydroxyethyl-methacrylate) (Langer et al., J. Biomed. Mater. Res., 15:167-277 (1981) and Langer, Chem. Tech., 12:98-105 (1982)), ethylene vinyl acetate (Langer et al., supra) or poly-D(−)-3-hydroxybutyric acid (EP 133,988). Sustained release compositions may also may include liposomes, which can be prepared by any of several methods known in the art. See e.g., Eppstein et al., Proc. Natl. Acad. Sci. USA, 82:3688-3692 (1985); EP 36,676; EP 88,046; 036,676; EP 088,046 and EP 143,949.

An effective amount of modulator composition will depend, for example, upon the therapeutic context and objectives. The appropriate dosage levels for treatment will thus vary depending, in part, upon the molecule delivered, the indication for which the modulator is being used, the route of administration, and the size (body weight, body surface or organ size) and condition (the age and general health) of the patient. A typical dosage may range from about 0.1 μg/kg to up to about 100 mg/kg or more, from about 1 μg/kg up to about 100 mg/kg; or 5 μg/kg up to about 100 mg/kg, depending on the factors mentioned above.

The frequency of dosing will depend upon the pharmacokinetic parameters of the modulator in the formulation used, which will typically be administered until a dosage is reached that achieves the desired effect. The composition may therefore be administered as a single dose, or as two or more doses (which may or may not contain the same amount of the desired molecule) over time, or as a continuous infusion via an implantation device or catheter. Further refinement of the appropriate dosage is routinely made by the clinician and is routinely performed by them. Appropriate dosages may be determined through use of appropriate dose-response data.

The routes of administration of the pharmaceutical composition contemplated include, for example, oral, injection by intravenous, intraperitoneal, intracerebral (intra-parenchymal), intracerebroventricular, intramuscular, intra-ocular, intraarterial, intraportal, or intralesional routes, or routes; sustained release systems or by implantation devices, bolus injection or continuously by infusion. Alternatively or additionally, the composition may be administered locally via implantation of a membrane, sponge, or other appropriate material on to which the modulator has been absorbed or encapsulated. Where an implantation device is used, the device may be implanted into any suitable tissue or organ, and delivery of the desired molecule may be via diffusion, timed-release bolus, or continuous administration.

### EXAMPLES

### MT1-MMP-Selective Regulation of WAT Development

Adipose tissue development was characterized in mice fed a standard 4% chow diet ad libitum. Mice used in these analyses included those harboring inactivating mutations in either MT1-MMP (Swiss Black background), MMP-2 (C57/BL6 background), MMP-9 (129Svev background) or MMP-3 [B10.RIIIH2r(71NS)/nMob background] as well as their respectie wild-type or littermate controls (Sabeh et al., 2004). At specified intervals, fat pads were isolated, weights determined, and tissues prepared for histologic analyses. H&E staining, transmission electron microscopy (TEM), and scanning electron microscopy (SEM) were performed as described (Hotary et al., 2003). Lipid staining was performed either with Oil Red-O (Sigma) or Nile Red (Sigma) (Zhang, et al., 2004) with confocal images obtained using a Carl Zeiss LSM 510 confocal microscope. Tissue collagen content was determined by Sirius Red staining (Sigma) (Filippov et al., 2005), and hydroxyproline content quantified as described (Sabeh et al., 2004). Immunofluorescent staining of degraded collagen was determined with a mouse monoclonal antibody raised against denatured type I collagen (Chun et al., 2004). Phosphorylated CREB was localized using an anti-p-CREB antibody (Upstate). TUNEL assay and PCNA staining were performed according to the respective manufacture's protocol (Roche and Santa Cruz), respectively (Hotary et al., 2003).

Dermal tissues isolated from 14-d old wild-type mice display a characteristic epidermal layer overlying a hair follicle- and adipocyte-rich hypodermis (Hausman et al., 2001). By contrast, while the MT1-MMP−/− epidermis and dermis exhibit normal overall structure, adipocytes are no longer visualized readily. At higher magnification, however, MT1-MMP-null adipocytes, with a cell diameter up to 10 times smaller than that found in wild-type adipocytes, can be discerned in association with nearby hair follicles. While the architecture of WAT deposits varies from tissue-to-tissue (e.g., Hausman et al., 2001), similar, if not identical effects on adipocyte size and WAT mass are observed in MT1-MMP−/− inguinal as well as epididymal fat pads. Further, whereas wild-type adipocytes are characterized by large lipid deposits as assessed by either Oil Red O staining or TEM, the MT1-MMP−/− adipocytes stain poorly and contain only small unilocular or multilocular deposits of fat. Though WAT expresses a range of other MMPs, including MMP-2, MMP-9 and MMP-3 (e.g., Chavey et al., 2003; Maquoi et al., 2003), male mice null for each of these proteinases do not display significant defects in adipocyte size or mass. Importantly, despite a failure to thrive, MT1-MMP−/− brown adipose tissue (BAT) appears normal relative to controls (interscapular mass of MT1-MMP+/+ and MT1-MMP−/− BAT is 0.67±0.5% and 0.61±0.07% of the respective body weights).

### MT1-MMP Exerts Global Effects on WAT Gene Expression and Function

To define functional consequences of the MT1-MMP-null status on WAT development, inguinal fat pads were isolated from 14-d old wild-type and MT1-MMP−/− mice, and gene expression characterized.

Total RNA from WAT (inguinal fat pads) or cultured preadipocytes was isolated from 14-d old MT1-MMP+/+ or MT1-MMP−/− mice (Trizol Reagent; Invitrogen) according to the Affymetrix technical manual. Hybridization, washing and staining of Affymetrix GeneChip® Mouse Expression Set 430 (Affymetrix) were performed at the University of Michigan Comprehensive Cancer Center (UMCCC) Affymetrix and cDNA Microarray Core Facility. For analysis, data were analyzed using the robust multi-array average (RMA) expression measure (Irizarry et al., 2003).

Unpaired and paired t-test were two-tailed and P<0.001 was considered statistically significant. A minimum two-fold difference in expression was adopted as the threshold for differential expression (Smyth et al., 2005). Gene ontology analysis was performed using GOstats packages (BioConductor) as described (Gentleman, 2004) Lipogenesis Assay After.a 12 h fast, adipocytes were isolated from the pooled inguinal fat pads of 14-d-old male MT1-MMP+/+ or MT1-MMP−/− mice following tissue digestion with collagenase type 3 (Worthington) as described (Marshall and Olefsky, 1980). Adipocytes (105 cells/ml) were resuspended in a buffer containing 5 mM D-glucose and 4 μM [U-14C]glucose (ICN Biomedicals) in the absence or presence of 100 nM insulin at 37° C. Following a 60 min incubation, 14C-incorporation into the lipid-soluble fraction was determined (Morales et al., 1992).

ELISAs for serum leptin (Crystal Chem Inc.), adiponectin (Linco Research) and albumin (Bethyl) levels in 14-d-old mice were performed according to the respective manufacturer's protocol.

Despite the phenotypic differences between wild-type and MT1-MMPnull WAT, more than 90% of the genes detected display no more than a 2-fold difference in expression. Furthermore, expression levels for key adipogenic transcription factors, including peroxisome proliferator-activated receptor γ (PPARγ) and sterol regulatory element binding protein I (SREBP-I) are largely unaffected as are levels for critical elements of the insulin signal transduction cascade (Supplement Table I) (e.g., Rosen and Spiegelman, 2000; Lazar, 2002).

While MT1-MMP has been reported to control the regulation of VEGF-A expression (Itoh and Seiki, 2006), a central factor in regulating WAT angiogenesis, MT1-MMP−/−tissues express normal levels of the transcript (Supplement Table I). However, mRNA levels for CCAAT/enhancer binding protein (C/EBP) family members, including C/EBPα and C/EBPβ, are depressed in tandem with the C/EBP-regulated target genes, leptin, aquaporin 7, steroyl CoA desaturase, PEPCK, and UCP-1 (Rosen and Spiegelman, 2000; Lazar, 2002). Further, gene sets linked to carbohydrate and lipid metabolism as well as mitochondrial function are inhibited strongly (Supplement Table I). Together, these data suggest that MT1-MMP deficiency exerts global effects on adipocyte structure and function by selectively controlling the expression of a significant subset of gene products critical to the adipogenic process.

Consistent with a gene expression profile that predicts selective effects on WAT function, insulin administered intraperitoneally to 14-d old mice elicits a comparable increase in Akt phosphorylation in both wild-type and null tissues in vivo (Saltiel and Kahn, 2001). However, downstream targets of the insulin signaling cascade are affected as MT1-MMP−/− adipocytes are unable to mount a normal lipogenic response. Further, whereas adiponectin levels are within normal range, circulating leptin is undetectable in MT1-MMP−/− mice. Functional defects in WAT deposits are also reflected in the induction of a lipodystrophic-like state in MT1-MMP−/− mice as evidenced by increases in both hepatic triglyceride and glycogen contents. Thus, MT1-MMP not only regulates adipocyte morphology, but also exerts control over a complex set of gene products that impact on lipid metabolism in vivo.

### MT1-MMP−/− Preadipocytes Retain Full Adipogenic Potential In Vitro

MT1-MMP is able to hydrolyze a wide variety of cell-associated targets ranging from integrins to growth factors (e.g., Lehti et al., 2005; Itoh and Seiki, 2006). To first determine whether MT1-MMP regulates adipogenesis via the hydrolysis of an adipocyte-derived target molecule, the differentiation of wild-type and MT1-MMP−/− preadipocytes to lipid-laden adipocytes was assessed in vitro. At postnatal day 3-5, WAT is not yet developed fully and primordial fat pads are populated largely by preadipocytes and their precursors (Hausman et al., 2001). Within this time frame, the inguinal fat pads of MT1-MMP+/+ and MT1-MMP−/− mice are morphologically indistinguishable with no changes in proliferation rates, apoptosis or vascular density.

Likewise, preadipocytes isolated from 3-5-d old wild-type or null tissues, and cultured in the absence of growth factors, display similar morphologies. In response to a mixture of pro-adipogenic factors (Zhang et al., 2004), wild-type preadipocytes differentiate and adopt a spherical shape, incorporate lipid, and express adipocyte markers. Surprisingly, MT1-MMP−/− preadipocytes likewise engage a normal adipogenic program to form mature adipocytes indistinguishable from controls.

### An In Vivo Cell Autonomous Defect in Adipocyte Differentiation

Adipose tissue is comprised of a complex mixture of adipocytes, endothelial cells, leukocytes and nerve cells (Napolitano, 1963; Hausman et al., 2001). As MT1-MMP is expressed widely in host tissues (Itoh and Seiki, 2006), in vivo defects in WAT development might potentially be assigned to the non-adipocyte compartment. Hence, taking advantage of the fact that preadipocytes can be induced to differentiate into mature adipocytes following in vivo transplantation (Mandrup et al., 1997), wild-type or MT1-MMP−/− stromal cells were transduced with a GFP expression vector and injected subcutaneously into the inguinal region of wild-type recipient mice.

Preadipocytes isolated from the inguinal fat pads of 4-d-old MT1-MMP-+/+ or MT1-MMP−/− mice were transduced with a GFP-retroviral expression vector and 2×10⁶ cells injected subcutaneously into 3-week-old Nu/Nu mice in either the substernal or inguinal regions (Mandrup et al., 1997). After 14 d, the mice were sacrificed and the tissues excised.

After a 14-d incubation period, GFP positive tissues recovered from mice inoculated with wild-type preadipocytes reveal the ectopic formation of well-developed adipocytes. The lipid-laden cells were positive for both GFP and the lipophilic dye, Nile Red. In marked contrast, only limited deposits of fat localizes to ectopic MT1-MMP-null adipocytes. Thus, while MT1-MMP−/− adipocyte precursors can differentiate in vitro, a cell autonomous defect is displayed in the in vivo setting.

### Dysregulated Collagen Deposition in MT1-MMP−/− WAT

The failure to recapitulate the MT1-MMP-null phenotype in vitro is consistent with the possibility that WAT development introduces an MT1-MMP target substrate that is unique to the in vivo environment. To this end, inguinal fat pad morphology was assessed by TEM. Indeed, while the ECM of normal WAT is known to be dominated by fibrillar type I collagen (Napolitano, 1963), the density of collagen fibrils in MT1-MMP−/− WAT was increased markedly with collagen levels elevated more than ten-fold. Interestingly, neither the collagen content of BAT nor tail tendon collagen content is affected by the MT1-MMP-null status of the mice. As MT1-MMP expresses pericellular collagenolytic activity (Chun et al., 2004; Sabeh et al., 2004), and type I collagen mRNA levels (as well as those of the other mouse MMP family members) are unaffected in MT1-MMP−/− WAT (Supplement Table II), the possibility was considered that MT1-MMP-type I collagen interactions control preadipocyte differentiation. Hence, wild-type or MT1-MMP−/− preadipocytes were cultured atop a bed of fibrillar type I collagen, and maturation induced with adipogenic factors. However, despite the presence of a type I collagen substratum, the MT1-MMP-null cells retain the ability to differentiate into mature adipocytes. Though the compliance or “stiffness” of the underlying substratum can regulate differentiation programs in a variety of cell types (Discher et al., 2005), altering collagen rigidity by varying gel thickness did not affect the adipogenic potential of wild-type or MT1-MMP−/− preadipocytes.

### A 3-D Collagen/MT1-MMP Axis Regulates Adipogenic Induction

In contrast with the cell-matrix interactions that occur at planar, 2-D interfaces, cell behavior is altered dramatically when cells are embedded within a 3-D matrix (Cukierrnan et al., 2002; Hotary et al., 2003; Beningo et al., 2004). Indeed, early studies of in vivo adipocyte development highlighted the fact that preadipocytes differentiate within—rather than atop—a 3-D matrix of type I collagen fibrils (Napolitano, 1963). To determine whether the modulatory effects of MT1-MMP on adipocyte differentiation are dependent on the 3-D architecture of the surrounding matrix, preadipocyte maturation was examined alternatively with cells dispersed and embedded within a fibrillar gel of type I collagen. Prior to the addition of the adipogenic cocktail, MT1-MMP+/+ preadipocytes cultured within a 3-D collagen matrix display a stellate morphology and organize an actin network marked by stress fibers. In contrast, the MT1-MMP−/− preadipocytes assume an abnormal, spiculated morphology wherein stress fibers were not discerned readily. Of note, the altered morphology of the MT1-MMP−/− preadipocytes was not observed when cells are cultured atop identically constructed collagen gels.

Cell shape and cytoskeletal architecture together conspire to regulate cellular responses to a range of exogenous stimuli (e.g., Chen et al., 1997; McBeath et al., 2004). As cells receive mechanical inputs from the surrounding matrix, downstream signaling pathways are activated that regulate cell geometry and the generation of RhoA-dependent tractional forces (McBeath et al., 2004; Discher et al., 2005). Whereas 3-D-embedded MT1-MMP+/+preadipocytes exert sufficient traction to rapidly contract released collagen gels, MT1-MMP-null cells are completely unable to remodel the matrix despite comparable levels of active RhoA. Defects in traction could conceivably alter lineage commitment of preadipocyte precursor cells (McBeath et al., 2004), but wild-type preadipocytes cultured in 3-D collagen in the presence of the endogenous MT1-MMP inhibitor, TIMP-2 (Chun et al., 2004; Sabeh et al., 2004), phenocopy the abnormal morphology and traction responses exhibited by MT1-MMP−/− preadipocytes. As predicted on the basis of their comparable shape and normal adipogenic potential under 2-D conditions, MT1-MMP+/+ preadipocytes (cultured in the absence or presence of TIMP-2) as well as their MT1-MMP−/− counterparts remodel collagen gels to equivalent degrees when cultured atop the 3-D matrices.

The abnormal set-point established by 3-D-embedded MT1-MMP−/− preadipocytes wherein cell shape and tension are perturbed raises the possibility that early responses to adipogenic growth factors may be altered. CREB (cAMP response element-binding protein) serves as a key transcriptional activator during adipocyte differentiation and undergoes rapid phosphorylation in response to stimulation with adipogenic hormones (Zhang et al., 2004). In 3-D gels, CREB is found in a constitutively phosphorylated state in both MT1-MMP+/+ and MT1-MMP−/− preadipocytes, but the levels of phosphorylated CREB (p-CREB) and total CREB were dramatically reduced in the null cells.

Consistent with an upstream defect in transcriptional regulation, microarray analyses of MT1-MMP−/− preadipocytes cultured with adipogenic factors under 3-D, but not 2-D, conditions in vitro recapitulated the in vivo profile of disturbed gene expression detailed in MT1-MMP-null WAT.

### Matrix Rigidity Controls MT1-MMP-Dependent Adipocyte Maturation

To assess the morphologic impact of MT1-MMP-deficiency on adipocyte development under 3-D culture conditions in vitro, cells isolated from wild-type and null littermates were embedded in type I collagen gels and adipocyte maturation monitored over a 6 d incubation period. Over this time course, wild-type preadipocytes express MT1-MMP and acquired the spherical shape characteristic of ap2-positive, adipocytes.

While proliferation rates of 3-D embedded MT1-MMP+/+ and MT1-MMP−/− preadipocytes were comparable under these conditions (i.e., at day 6, cell number increased from 2.0×10⁵ to 7.4±1.2×10⁵ and 8.9±1.1×10⁵, respectively, for wild-type and null cells), MT1-MMP−/− adipocytes adopted a shrunken morphology similar to that observed in vivo and fail to accumulate lipid deposits comparably to wild-type cells.

The abnormal morphology and differentiation of MT1-MMP−/− preadipocytes cultured in 3-D collagen supports a model wherein wild-type preadipocytes mobilize MT1-MMP to degrade surrounding collagen in a fashion that coordinately alters matrix compliancy and cell shape. Indeed, while the hypertrophic growth of MT1-MMP+/+ adipocyted coincides with the accumulation of pericellular type I collagen degradation products, matrix remodeling was absent in MT1-MMP−/− cells. Together, these findings suggest that MT1-MMP-dependent collagenolysis plays a required role in regulating pericellular matrix rigidity. Indeed, the 3-D differentiation of wild-type preadipocytes was suppressed markedly as collagen density, and hence, rigidity, is increased. Conversely, the phenotype of MT1-MMP−/− preadipocytes was normalized partially when the type I collagen concentration was reduced 3-fold to 0.8 mg/ml. Similarly, leptin secretion increased from undetectable levels to approximately 40% of those found in 3-D cultures of MT1-MMP+/+adipocytes (i.e., 76 pg/ml versus ˜200 pg/ml for wild-type cells cultured in 2.4 μg/ml collagen). A required role for MT1-MMP during adipocyte differentiation was restricted to 3-D type I collagen as culturing MT1-MMP−/− preadipocytes within a rigid, 3-D gel of cross-linked fibrin (a fibrillar ECM component that is degraded readily by either adipocyte MT1-MMP or MT3-MMP; Hotary et al., 2002; Filippov et al., 2005) is permissive for null cell differentiation into lipid-laden adipocytes that recover the bulk of their leptin secretory activity.

Likewise, MT1-MMP−/− preadipocytes embedded in Matrigel, a basement membrane-like composite that can be remodeled independently of MT1-MMP (Hotary et al., 2002; Filippov et al., 2005), also retained adipogenic potential. Finally, to assess the ability of the MT1-MMP enzyme to rescue the MT1-MMP−/− phenotype, as well as the structural requirements underlying MT1-MMP-dependent adipogenesis, MT1-MMP-null preadipocytes were transduced with a series of MT1-MMP retroviral expression vectors immediately prior to 3-D culture. Under these conditions, the developmental defects displayed by MT1-MMP−/− preadipocytes cultured in 3-D collagen were reversed following retroviral transduction of wild-type MT1-MMP, but not following transduction with either a catalytically inactive mutant or a transmembrane-deleted, soluble form of the active enzyme. Hence, within the confines of a 3-D collagen matrix, membrane-tethered, catalytically active MT1-MMP played a required role in regulating preadipocyte differentiation.

### MT1-MMP Regulates WAT Collagen Architecture In Vivo

Taken together, these results provide a model wherein MT1-MMP-dependent remodeling of the primordial fat pad network of type I collagen fibrils plays a key role in regulating WAT development in vivo. As such, inguinal fat pads were isolated from 4- and 14-d old MT1-MMP+/+ or MT1-MMP−/− mice, and in situ collagen degradation as well as collagen architecture assessed. The development of the primordial fat pad of 4-d old MT1-MMP+/+ mice was marked by collagen degradation as well as p-CREB nuclear staining. By postnatal day 14, the collagenolytic phase of WAT development observed in wild-type mice subsided, and SEM analysis demonstrated that both the exterior and interior faces of the WAT were decorated with a delicate network of fibrillar collagen that invests the mature adipocytes. In marked contrast, at postnatal day 4, the MT1-MMP−/− inguinal fat pad displayed only minimal collagen degradation despite an interstitial collagen content similar to that observed in controls. Further, in the absence of type I collagen remodeling, p-CREB was only weakly expressed in the null tissues. Consequently, by postnatal day 14, a time point at which type 1 collagen mRNA expression levels were comparable to wild-type tissues (Supplement Table II), MT1-MMP−/− preadipocytes remained entangled in a strikingly dense meshwork of fibrillar collagen. Hence, MT1-MMP plays a required role in regulating WAT matrix turnover and the subsequent development of a structurally intact, functional fat pad.

### MT1-MMP-Dependent Regulation of 3-D Matrix Rigidity

The morphologic and functional maturation of WAT is initiated almost immediately after birth wherein mesenchymal cell precursors commit to the adipocyte lineage and engage a complex transcription factor cascade that leads to the generation of lipid-laden fat cells (Napolitano, 1963; Rosen and Spiegelman, 2000; Hausman et al., 2001). Herein, it is demonstrated that MT1-MMP-null mice harbor a WAT-specific lipodystrophic phenotype which manifests itself as a consequence of a requirement for the membrane anchored proteinase in regulating adipocyte differentiation within the confines of the type I collagen-rich, 3-D ECM that dominates the developing fat pad. MT1-MMP Directs the WAT Transcriptome Current models support a developmental program wherein a series of transcription factors, including Krox20, KLF5, C/EPBβ and C/EPBδ, act as early initiators of a highly regulated process that ultimately controls the expression of C/EPBα and PPARγ which, in turn, function as the terminal effectors of adipogenesis (Rosen and Spiegelman, 2000; Lazar, 2002; Gonzalez, 2005). Consistent with the fact that MT1-MMP−/− adipocytes are able to store lipid, albeit in small quantities, PPARγ mRNA levels were normal, as were the expression of well-characterized downstream target genes, including aP2 and LPL (Rosen and Spiegelman, 2000; Lazar, 2002). Nonetheless, an analysis of the full WAT transcriptome revealed a perturbed gene expression profile with marked reductions in transcripts linked to carbohydrate metabolism, fatty acid synthesis and mitochondrial biogenesis (Rosen and Spiegelman, 2000; Lazar 2002). These findings are consistent with the significant reductions registered in the expression of multiple transcription factors, including C/EPBβ, C/EPBa, PPARα and NRFB1 that all serve to modulate PPARγ-regulated adipogenic programs (Rosen and Spiegelman, 2000; Lazar, 2002). Together, the multiplicity of downstream targets affected by MT1-MMP deficiency coalesces to undermine WAT development.

To determine the molecular mechanisms by which MT1-MMP regulates adipogenesis, preadipocyte maturation was assessed in a well-characterized in vitro model (Zhang et al., 2004).

Preadipocytes were isolated from the inguinal fat pads of 4-d-old MT1-MMP+/+ or MT1-MMP−/− mice as described (Mitchell et al., 1997). Preadipocytes (2×10⁵) were then cultured in Falcon 12-well plates in which the tissue culture plastic was either left uncoated or pre-coated with 200-500 μA of a 2.4 mg/ml solution of native, acid-extracted type I collagen (Chun et al., 2004; Sabeh et al., 2004). Alternatively, preadipocytes were embedded in a constrained/attached, type I collagen gel (500 μl of a 0.8 mg/ml, 1.6 mg/ml, 2.4 mg/ml or 3.2 mg/ml solution as indicated) (Hotary et al., 2003; Sabeh et al., 2004). In selected experiments, MT1-MMP−/− preadipocytes were transduced with retroviral MT1-MMP expression vectors encoding the wild-type proteinase, a catalytically-inactive mutant harboring an E→A240 substitution in the catalytic domain or a proteolytically active, transmembrane-deletion mutant (Chun et al., 2004). Adipocyte differentiation was induced with a mixture of 10 μg/ml insulin, 0.5 mM 3-isobutyl-1-methylxanthine, and 0.25 μM dexamethasone (all from Sigma) (Zhang et al., 2004). Five to 7 d post-stimulation, samples were fixed in 4% paraformaldehyde and stained with both Nile Red (Sigma) and Hoechst 33342 (Molecular Probe) for visualization of intracellular lipid deposits and nuclei, respectively. The ability of cultured preadipocytes to contract Collagen gels was monitored as described previously (Sabeh et al., 2004). GTP-RhoA, RhoA, ROCK, CREB, p-CREB, MT1-MMP and ap2 protein levels were determined as described (McBeath et al., 2004; Zhang et al., 2004; Lehti et al., 2005).

Unexpectedly, and in marked contrast to the in vivo phenotype, MT1-MMP−/− preadipocytes cultured in vitro under standard 2-D conditions were able to develop normally into lipid-laden adipocytes despite the fact that the functional status of these cells remains impaired following transplantation into wild-type mice. Given these findings, attention was focused on early studies of WAT development wherein preadipocyte differentiation and maturation in vivo were reported to proceed within a dense network of interwoven type I collagen fibrils (Napolitano, 1963). Initially, the possibility was considered that the MT1-MMP-dependent remodeling of an underlying collagen substratum might alone regulate preadipocyte differentiation by either affecting cell shape, matrix rigidity or cell tension (McBeath et al., 2004; Discher et al., 2005; Paszek et al., 2005). However, following culture atop a 3-D substratum of type I collagen fibrils, MT1-MMP−/− preadipocytes retained the ability to differentiate comparably to wild-type cells. Hence, local changes in matrix rigidity, matrix adhesion or cell traction—at least under 2-D culture conditions—are unlikely to play major roles in the MT1-MMP-dependent adipogenic program. Instead, the in vivo phenotype of MT1-MMP−/− adipocytes could only be recapitulated when preadipocytes were embedded within a 3-D matrix of type I collagen fibrils. To date, the functional significance of the preadipocyte-3-D type I collagen axis has not been generally considered (Hilliou et al., 1988), but recent studies from have emphasized the ability of MT1-MMP to function as the dominant pericellular collagenase in mesenchymal cells (Chun et al., 2004; Sabeh et al., 2004). Consequently, it was proposed that adipocytes might mobilize the proteinase to remodel the surrounding matrix in an effort to control cell shape, matrix compliancy and/or cell tension in the third dimension (e.g., Cukierman et al., 2002; Tan et al., 2003; Discher et al., 2005). Hence, data herein indicate that wildtype preadipocytes form lipid-laden adipocytes in 3-D culture as a direct consequence of their ability to mobilize MT1-MMP and proteolytically remodel the surrounding collagen matrix. By contrast, MT1-MMP−/− adipocyte differentiation is aborted—in vitro as well as in vivo—when null cells find themselves physically constrained within the interstices of the surrounding type I collagen network. In 3-D culture, it was posited that in the absence of establishing an optimal cell shape and level of cytoskeletal tension with the surrounding matrix of native and proteolyzed collagen, signaling cascades critical to the normal adipogenic program fail to engage properly. Interestingly, despite a required role for MT1-MMP in regulating the type I collagen mass in WAT, brown fat was spared, presumably as a consequence of its low content of interstitial collagen (Barnard, 1969).

### Cell Shape, Signaling and the Nuclear Matrix

Given the complex morphologic and functional consequences for WAT development following the loss of MT1-MMP, it seems reasonable to query as to how interactions between preadipocytes and the surrounding collagen matrix could assume such global importance in regulating adipocyte biology. As changes in 3-D cell volume and geometry may influence transcriptional as well as post-transcriptional machinery (Cukierman et al., 2002; Discher et al., 2005), no single defect may fully underlie the 3-D collagen-specific changes exhibited by MT1-MMP−/− preadipocytes.

These current studies have demonstrated the role of MT1-MMP during development, and that the expression of the membrane-anchored protease is also regulated postnatally in response to fat intake (Lijnen et al., 2002; Chavey et al., 2003). Indeed, high fat diets have been reported to induce local losses in the type I collagen content of affected fat pads, and mice expressing lower levels of type I collagen can display hypertrophic WAT (Lijnen et al., 2002; Bradshaw et al., 2003). As such, MT1-MMP acts as a new proteolytic “rheostat” that controls WAT transcriptional activity and function by remodeling the surrounding ECM barriers as the adipocyte accumulates or metabolizes lipids in response to the changing energy demands of the host.

The foregoing description is given for clearness of understanding only, and no unnecessary limitations should be understood therefrom, as modifications within the scope of the invention may be apparent to those having ordinary skill in the art.

### REFERENCES

References cited herein are incorporated by reference in their entireties.
Alexander, C. M., Selvarajan, S., Mudgett, J., and Werb, Z. (2001). Stromelysin-1 regulates adipogenesis during mammary gland involution. J. Cell Biol. 152, 693-703.
Barnard, T. (1969). The ultrastructural differentiation of brown adipose tissue in the rat. J. Ultrastruct. Res. 29, 311-332.
Beningo, K. A., Dembo, M., and Wang, Y. (2004). Responses of fibroblasts to anchorage of dorsal extracellular matrix receptors. Proc. Natl. Acad. Sci. USA 101, 18024-18029.
Bouloumie, A., Sengenes, C., Portolan, G., Galitzky, J., and Lafontan, M. (2001). Adipocyte produces matrix metalloproteinases 2 and 9. Involvement in adipose differentiation. Diabetes 50, 2080-2086.
Bradshaw, A. D., Graves, D. C., Motamed, K., and Sage, E. H. (2003). SPARC-null mice exhibit increased adiposity without significant differences in overall body weight. Proc. Natl. Acad. Sci. USA 100, 6045-6050.
Chavey, C., Mari, B., Monthouel, M. N., Bonnafous, S., Anglard, P., Van Obberghen, E., and Tartare-Deckert, S. (2003). Matrix metalloproteinases are differentially expressed in adiose tissue during obesity and modulate adipocyte differentiation. J. Biol. Chem. 278, 11888-11896.
Chen, C. S., Mrksich, M., Huang, S., Whitesides, G. M., Ingber, D. E. (1997) Geometric control of cell life and death. Science 276:1425-1428.
Chun, T. H., Sabeh, F., Ota, I., Murphy, H., McDonagh, K., Holmbeck, K., Birkedal-Hansen, H., Allen, E. D., and Weiss, S. J. (2004). MT1-MMP-dependent neovessel formation within the confines of the 3-dimensional extracellular matrix. J. Cell Biol. 167, 757-767.
Croissandeau, G., Chretien, M., and Mbikay, M. (2002). Involvement of matrix metalloproteinases in the adipose conversion of 3T3-L1 preadipocytes. Biochem. J. 364, 739-746.
Cukierman, E., Pankov, R, and Yamada, K. M. (2002). Cell interactions with three-dimensional matrices. Curr Opin. Cell Biol. 14, 633-639.
Discher, D. E., Janmey, P., and Wang, Y. L. (2005) Tissue cells feel and respond to the stiffness of their substrate. Science 310:1139-1143.
Filippov, S., Koenig, C. C., Chun, T. H., Hotary, K. B., Ota, I., Bugge, T. H., Roberts, J. D., Fay, W. P., Birkedal-Hansen, H., Holmbeck, K., et al. (2005). MT1-matrix metalloproteinase directs arterial wall invasion and neointima formation by vascular smooth muscle cells. J. Exp. Med. 202:663-671.
Flier, J. S. (2004). Obesity wars: Molecular progress confronts an expanding epidemic. Cell 116, 337-350.
Gentleman, R. (2004) Using GO for statistical analyses. In COMPSTAT 2004—Proceedings in Computational Statistics. J. Antoch, ed. (Publisher: Physica Verlag), pp. 171-180.
Gonzalez, F. J. (2005). Getting fat: Two new players in molecular adipogenesis. Cell Metab. 1, 85-86.
Hausman, D. B., DiGirolamo, M., Bartness, T. J., Hausman, G. J., and Martin, R. J. (2001). The biology of white adipocyte proliferation. Obes. Rev. 2, 239-254.
Hilliou, F., Pairault, J., Dominice, J., and Redziniak, G., (1988). Growth and differentiation of 3T3-F442A preadipocytes in three-dimensional gels of native collagen. Exp. Cell Res. 177, 372, 381.
Hiraoka, N., Allen, E., Apel, I. J., Gyetko, M. R., and Weiss, S. J. (1998). Matrix metalloproteinases regulate neovascularization by acting as pericellular fibrinolysins. Cell 95, 365-377.
Holmbeck, K., Bianco, P., Caterina, J., Yamada, S., Kromer, M., Kuznetsov, S. A., Mankani, M., Robey, P. G., Poole, A. R., Pidoux, I., et al. (1999). MT1-MMP-deficient mice develop dwarfism, osteopenia, arthritis, and connective tissue disease due to inadequate collagen turnover. Cell 99, 81-92.
Hotary, K. B., Yana, I., Sabeh, F., Li, X-Y., Holmbeck, K., Birkedal-Hansen, H., Allen, E. D., Hiraoka, N., and Weiss, S. J. (2002). Matrix metalloproteinases regulate fibrininvasive activity via MT1-MMP-dependent and independent processes. J. Exp. Med. 195, 295-308.
Hotary, K. B., Allen, E. D., Brooks, P. C., Datta, N. S., Long, M. W., and Weiss, S. J. (2003). Membrane type I matrix metalloproteinase usurps tumor growth control imposed by the three-dimensional extracellular matrix. Cell 114, 33-45.
Irizarry, R. A., Hobbs, B., Collin, F., Beazer-Barclay, Y. D., Antonellis, K. J., Scherf, U., and Speed, T. P. (2003). Exploration, normalization, and summaraies of high density oligonucleotide array probe level data. Biostatistics 4, 249-264.
Itoh, Y., and Seiki, M., (2006). MT1-MMP: A potent modifier of pericellular microenvironment. J. Cell. Phys. 206, 1-8.
Lazar, M. A. (2002). Becoming fat. Genes Dev. 16, 1-5.
Lehti, K., Allen, E., Birkedal-Hansen, H., Holmbeck, K., Miyake, Y., Chun, T. H., and Weiss, S.J. (2005). An MT1-MMP-PDGF receptor-β axis regulates mural cell investment of the microvasculature. Genes Dev. 19, 979-991.
Lijnen, H. R., Maquoi, E., Demeulemeester, D., Van Hoef, B., and Collen, D. (2002). Modulation of fibrinolytic and gelatinolytic activity during adipose tissue development in a mouse model of nutritionally induced obesity. Thromb. Haemost. 88, 345-353.
Lilla, J., Stickens, D., and Werb, Z. (2002). Metalloproteases and adipogenesis: A weighty subject. Am. J. Pathol. 160, 1551-1554.
Mandrup, S., Loftus, T. M., MacDougald, O. A., Kuhajda, F. P., and Lane, M.D. (1997). Obese gene expression at in vivo levels by fat pads derived from s.c. implanted 3T3-F442A preadipocytes. Proc. Natl. Acad. Sci. USA 94, 4300-4305.
Maquoi, E., Demeulemeester, D., Voros, G., Collen, D., and Lijnen, H.R. (2003). Enhanced nutritionally induced adipose tissue development in mice with stromelysin-I gene inactivation. Thromb. Haemost. 89, 696-704.
Marshall, S., and Olefsky, J. M. (1980). Effects of insulin incubation on insulin binding, glucose transport, and insulin degradation by isolated rat adipocytes. Evidence for hormone-induced desensitization at the receptor and postreceptor level. J. Clin. Invest. 66, 763-772.
McBeath, R., Pirone, D. M., Nelson, C. M., Bhadriraju, K., and Chen, C. S. (2004). Cell shape, cytoskeletal tension, and RhoA regulate stem cell lineage commitment. Dev. Cell 6, 483-495.
Morales, L. M., Campos, G., Ryder, E., and Casanova, A. (1992). Insulin and lipogenesis in rat adipocytes. I. Effect of insulin incubation on lipid synthesis by isolated rat adipocytes. Biochem. Biophys. Res. Commun. 188, 807-812.
Morroni, M., Giordano, A., Zingaretti, M. C., Boiani, R., De Matteis, R., Kahn, B. B., Nisoli, E., Tonello, C., Pisoschi, C., Luchetti, M., et al. (2004). Reversible transdifferentiation of secretory epithelial cells into adipocytes in the mammary gland. Proc. Natl. Acad. Sci. USA 101, 16801-16806.
Napolitano, L. (1963). The differentiation of white adipose cells. An electron microscope study. J. Cell Biol. 18, 663-679.
Paszek, M. J., Zahir, N., Johnson, K. R., Lakins, J. N., Rozenberg, G. I., Gefen, A., Reinhart-King, C. A., Margulies, S. S., Dembo, M., Boettiger, D., et al. (2005) Tensional homeostasis and the malignant phenotype. Cancer Cell 8:241-254.
Rosen, E. D., and Spiegelman, B. M. (2000). Molecular regulation of adipogenesis. Annu. Rev. Cell Dev. Biol. 16, 145-171.
Sabeh, F., Ota, I., Holmbeck, K., Birkedal-Hansen, H., Soloway, P., Balbin, M., Lopez-Otin, C., Shapiro, S., Inada, M., Krane, S., et al. (2004). Tumor cell traffic through the extracellular matrix is controlled by the membrane-anchored collagenase, MT1-MMP. J. Cell Biol. 167, 769-781.
Saltiel, A. R., and Kahn, C. R. (2001). Insulin signaling and the regulation of glucose and lipid metabolism. Nature 414, 799-806.
Smyth, G. K., Michaud, J., and Scott, H. S. (2005). Use of within-array replicate spots for assessing differential expression in microarray experiments. Bioinformatics. 21, 2067-2075.
Spiegelman, B. M., and Ginty, C. A. (1983). Fibronectin modulation of cell shape and lipogenic gene expression in 3T3-adipocytes. Cell 35,657-666.
Tan, J. L., Tien, J., Pirone, D. M., Gray, D. S., Bhadriraju, K., and Chen, C. S. (2003). Cells lying on a bed of microneedles: An approach to isolate mechanical force. Proc. Natl. Acad. Sci. USA 100, 1484-1489.
Yamauchi, T., Oike, Y., Kamon, J., Waki, H., Komeda, K., Tsuchida, A., Date, Y., Li, M. X., Miki, H., Akanuma, Y., et al. (2002) Increased insulin sensitivity despite lipodystrophy in Crebbp heterozygous mice. Nat. Genet. 30, 221-226.
Zhang, J. W., Klemm, D. J., Vinson, C., and Lane, M. D. (2004). Role of CREB in transcriptional regulation of CCAAT/enhancer-binding protein β gene during adipogenesis. J. Biol. Chem. 279:4471-4478.
Zhou, Z., Apte, S. S., Soininen, R., Cao, R., Baaklini, G. Y., Rauser, R. W., Wang, J., Cao, Y., and Tryggvason, K. (2000). Impaired endothondral ossification and angiogenesis in mice deficient in membrane-type matrix metalloproteinase I. Proc. Natl. Acad. Sci. USA 97, 4052-4057.
Fugita, et al., J Nat Prod. (2003) 66:569-71.
Toth, et al., J Biol Chem. (2000) 275:41415-23.
Toth, et al., J. Biol. Chem., (2002). 277:26340-26350
That, et al., Cancer Research (2005) 65:6543-6550
Yamamoto, et al., J. Med. Chem. (1998), 41:1209-1217
Muñoz-Nájar, et al., Oncogene (2006) 25:2379-2392.
Rosenthal, et al., Molecular Cancer Research (2005) 3:195-202
Hurst, et al., Biochem. J. (2005) 392:527-536

What is claimed is:

1. A methods of regulating fat storage comprising the step of administering a modulator of membrane-tethered metalloproteinase 1 (MT1-MMP) in an amount effective to modulate MT1-MMP activity.

2. The method of claim 1 wherein the modulator is an inhibitor of MT1-MMP and fat storage in inhibited.

3. The method of claim 1 wherein the modulator is an activator of MT1-MMP and fat storage is increased.

4. A method of increasing fat storage comprising the step of administering an amount of membrane-tethered metalloproteinase 1 (MT1-MMP) in an amount effective to increase fat storage.

5. A method of regulating carbohydrate metabolism comprising the step of administering a modulator of membrane-tethered metalloproteinase 1 (MT1-MMP) in an amount effective to modulate carbohydrate metabolism.

6. The method of claim 5 wherein the modulator is an inhibitor of MT1-MMP and carbohydrate metabolism in inhibited.

7. The method of claim 5 wherein the modulator is an activator of MT1-MMP and carbohydrate metabolism is increased.

8. A method of increasing carbohydrate metabolism comprising the step of administering an amount of membrane-tethered metalloproteinase 1 (MT1-MMP) in an amount effective to increase carbohydrate metabolism.

9. A method of regulating fatty acid synthesis comprising the step of administering a modulator of membrane-tethered metalloproteinase 1 (MT1-MMP) in an amount effective to modulate fatty acid synthesis.

10. The method of claim 9 wherein the modulator is an inhibitor of MT1-MMP and fatty acid synthesis in inhibited.

11. The method of claim 9 wherein the modulator is an activator of MT1-MMP and fatty acid synthesis is increased.

12. A method of increasing fatty acid synthesis comprising the step of administering an amount of membrane-tethered metalloproteinase 1 (MT1-MMP) in an amount effective to increase fatty acid synthesis.

13. A method of regulating fat storage comprising the step of administering a modulator of a membrane-tethered metalloproteinase 1 (MT1-MMP) downstream pathway component in an amount effective to modulate activity of said component.

14. The method of claim 13 wherein the downstream pathway component is one or more transcription factors.

15. The method of claim 14 wherein the one or more transcription factors is selected from the group consisting of C/EPBβ, C/EBPα, PPARα AND NRFB1.

16. The method of claim 13 wherein the modulator increases activity of said downstream pathway component

17. The method of claim 13 wherein the modulator decreases activity of said downstream pathway component.
